(19) United States
(12) Patent Application Publication
(10) Pub. No.: US 2012/0079625 A1
(43) Pub. Date: Mar. 29, 2012

(54) Method for protecting living plants from harmful insects via a sheetlike structure

(75) Inventors: Thomas ZÜEHLKE, Solingen (DE); Lars WEICHEL, Maikammer (DE); Michael HABERMANN, Bernshausen (DE); Christian TOMICZEK, Wien (AT)

(73) Assignee: BASF SE, Ludwigshafen (DE)

(21) Appl. No.: 13/238,075

(22) Filed: Sep. 21, 2011

Related U.S. Application Data
(60) Provisional application No. 61/385,570, filed on Sep. 23, 2010.

(51) Int. Cl. A01H 5/00; A01N 53/14; A01N 43/36; B32B 3/06; A01N 43/56

(52) U.S. Cl. 800/298; 428/100; 428/99; 514/407; 514/427; 514/531

(57) ABSTRACT

The present invention provides a method for protecting living plants from harmful insects by using a sheetlike structure impregnated with an insecticide to cover the surface of the trunk, pseudotrunk, branch, root ball and/or root region of the plant. Additionally provided are living plants whose trunk, pseudotrunk, branch, root ball and/or root region surface is covered with a sheetlike structure impregnated with an insecticide. The invention also relates to a rectangular sheetlike structure impregnated with an insecticide and comprising a fastening means which, following tubular coverage of the surface of trunk, pseudotrunk or branch of a plant, allows a permanent hold thereto. It further relates to a sheetlike structure impregnated with an insecticide, in the form of a perforated sheet which has a continuous interruption between the outer and inner edges. Lastly, it also relates to the use of the sheetlike structure for protecting living plants from harmful insects.

The present invention provides a method for protecting living plants from harmful insects by using a sheetlike structure impregnated with an insecticide to cover the surface of the trunk, pseudostem, branch, root ball and/or root region of the plant. Additionally provided are living plants whose trunk, pseudostem, branch, root ball and/or root region surface is covered with a sheetlike structure impregnated with an insecticide. The invention also relates to a rectangular sheetlike structure impregnated with an insecticide and comprising a fastening means which, following tubular coverage of the surface of trunk, pseudostem or branch of a plant, allows a permanent hold thereto. It further relates to a sheetlike structure impregnated with an insecticide, in the form of a perforated disk which has a continuous interruption between the outer and inner edges. Lastly, it also relates to the use of the sheetlike structure for protecting living plants from harmful insects. Combinations of preferred features with other preferred features are encompassed by the present invention.

Various insects require living plant parts for their reproduction and/or development. For example, bark beetles and bast bark beetles enter living plant plants in order to lay eggs there. Certain species of insects, such as plant-damaging butterflies, longhorn beetles and flathead beetles, lay their eggs on parts of plants, and the hatching larvae enter woody, living parts of plants to continue their development. Other insects eat leaves, needles or bark. In the case of cork oaks, the valuable cork should be protected from the penetration of the cork oak bark beetle. Various insects overwinter in the bark of living trees, and in the spring migrate to the crowns of the trees to feed. In the root region itself and around the plants, furthermore, active pests live (such as citrus longhorn beetles, cockchafers, sawflies, and weevils), the object being to hinder the hatching and/or adult emergence of these pests, and also their feeding on bark and trunk.

The plants and plant parts to be protected have hitherto been sprayed directly with an insecticide. The disadvantages lie above all in the drift (precise-placement application not possible) and hence in the contamination of the close vicinity. For this reason, many techniques cannot even be employed in water protection regions or during flowering, in order to protect bees, including bumblebees. Moreover, on application of the existing techniques, whole trees and shrubs are treated, and large volumes of insecticides are delivered.

An object of the present invention was to find a method for protecting living plants, such as trees and shrubs, from harmful insects.

The object has been achieved by means of a method for protecting living plants from harmful insects by using a sheetlike structure impregnated with an insecticide to cover the surface of the trunk, pseudostem, branch, root ball and/or root region of the plant.

Living plants are plants which are biologically active and intact. They differ from dead plants, which are no longer able to grow. The roots of living plants are typically in contact with substrate, such as with the earth or with substrate in pots, for example. Trees in a state of dormancy, such as in winter, for example, are also considered to be living plants. Plants with exposed roots as well, such as trees with exposed roots, may be living plants, provided they are biologically active and intact.

Suitable plants are all those which have at least one trunk, pseudostem or branch. In addition, suitable plants are those having a root ball of at least 3 cm (preferably at least 10 cm, and more preferably at least 30 cm) in diameter, examples being trees and shrubs. Additionally suitable as plants are those having a root region of at least 20 cm in diameter (preferably at least 50 cm), examples being trees and shrubs.

Preferred plants are trees, such as coniferous trees (such as pine, spruce, Douglas fir, larch, stone pine, fir, cedar, Swiss pine) and deciduous trees (such as maple, acacia, birch, pear, beech, oak (such as cork oak), elder, aspen, ash, wild service, hazel, hornbeam, cheery, chestnut, lime, American walnut, poplar, plane, peach, olive, robinia, elm, walnut, gum, zebrano, willow, turkey oak). Particularly preferred plants are deciduous trees.

Preferred deciduous trees are fruit trees, such as those of pome fruit (e.g., apple, pear, quince, sorb apple [Sorbus domestica]), stone fruit (e.g., cherry, sour cherry, plum, quetsch, peach, apricot, nectarine), or soft fruit cultivated in tree form (e.g., blackcurrants, gooseberries), with pome fruit and stone fruit being preferred. In a further embodiment, oaks, such as cork oaks, are preferred deciduous trees.

The trunk, pseudostem, and branch, as plant organs are known to the skilled person. Parts of a plant that are shaped anatomically like a trunk are also considered to be pseudostem. The root ball may have its roots exposed, or the roots may be surrounded with substrate. The diameter of trunk, pseudostem, and branch is usually at least 0.5 cm, preferably at least 2 cm, and more preferably at least 5 cm. The root region of a plant is understood typically to be the region in the vicinity of the plant within which its roots spread out. It is preferably an approximately circular region around the plant on the soil surface.

In one preferred embodiment, in the method of the invention, the sheetlike structure is used to cover the surface of the trunk, pseudostem and/or branch (more particularly the trunk or pseudostem, especially the trunk) of the plant.

In another preferred embodiment, in the method of the invention, the sheetlike structure is used to cover the surface of the root ball of the plant.

In another preferred embodiment, in the method of the invention, the sheetlike structure is used to cover this surface of the root region of the plant.

The sheetlike structure covers the surface of the trunk, pseudostem, branch, root ball and/or root region. The term “cover” here means that the sheetlike structure has at least partial areal contact with the surface.

The sheetlike structure covers the surface, in the method of the invention, for a duration of at least one week, preferably at least one month, more preferably at least three months, very preferably at least six months, and especially at least 12 months. The sheetlike structure may cover the surface for up to 10 years, preferably up to 3 years.

Examples of suitable sheetlike structures include textile materials (e.g., a cotton or polyester fabric), nontextile plastics materials, paper, leather, synthetic leather, polymeric film (made, for example, of polypropylene, polyethylene, polystyrene, or cellulose), and other, preferably flexible, materials.

The sheetlike structure typically has an area of at least 0.001 m², preferably of at least 0.005 m², and more preferably of at least 0.05 m². The area can be up to 100.0 m², preferably up to 30.0 m², more preferably up to 3.0 m².

The sheetlike structure used is preferably a textile material, more particularly nets of textile fibers. These may be nets of natural fibers or of synthetic fibers. The fibers in question may of course also comprise blends of two or more different fibers. Examples of natural fibers include cotton, jute or linen fibers. Preference is given to synthetic fibers made from suitable polymers. Examples include polyamides, polyesters, polyacrylonitrile or polyolefins. Preference is given to polyamides, polyolefins, and polyesters, more preferably polyolefins, especially polypropylene or polyethylene, and polyesters. Especially preferred are polyester fibers, more particularly polyethylene terephthalate (PET).

The fibers may be monofilaments, oligofilaments or multifilaments, and may be smooth or textured.

In the case of polypropylene and polyethylene, the polymers in question may be polypropylene or polyethylene homopolymers. Alternatively they may be copolymers which in addition to the ethylene or propylene, respectively, comprise small amounts of other comonomers. Suitable comonomers may in particular be other olefins such as, for example, ethylene or propylene, and also 1-butene, 2-butene, isobutene, 1-pentene, 1-hexene, 1-heptene, 1-octene, styrene or α-methylstyrene, dienes and/or polyenes. The fraction of comonomers in the polyethylene or polypropylene is generally not more than 20% by weight, preferably not more than 10% by weight. The nature and amount of the comonomers are selected by the skilled person in accordance with the desired fiber properties.

Particularly preferred for fiber making are viscous products of relatively high molecular mass which are characterized, conventionally, by their melt flow index (determined in accordance with ISO 1133). A material may preferably be at least one polypropylene or polyethylene having a melt flow index MFR (230° C., 2.16 kg) of 0.1 to 60 g/10 min. It is preferably polypropylene having a melt flow index MFR (230° C., 2.16 kg) of 1 to 50 g/10 min, more preferably 10 to 45 g/10 min, and, for example, 30 to 40 g/10 min. Polypropylene grades of this kind are particularly suitable for producing fibers. Of course, a mixture of two or more different grades of polypropylene may also be employed.

Depending on the type of net, the textile fibers have a thickness of 0.05 to 0.6 mm, preferably 0.1 mm to 0.4 mm, more preferably 0.12 to 0.35 mm, and very preferably 0.2 to 0.3 mm.

The nets that are preferably used preferably have a pattern of meshes with an even number of corners. These nets may consist preferably of only one single kind of meshes, as for example only of tetragonal meshes or only of hexagonal meshes, or they may also include two or more kinds of different meshes, such as a combination of octagonal and tetragonal meshes, for example.

The meshes of the net ought here preferably to be substantially of the same type—in other words, while the net may indeed feature minor deviations in respect of shape and size of the meshes, the values will not vary unduly around the mean values.

The mesh size of the net may vary widely, being, for example, from 0.5 mm to 25 mm, preferably from 1 mm to 20 mm. Preferred mesh sizes (length of the side of a square mesh) are in the range from 5 mm, preferably 2.5 mm, more particularly 1.5 mm, as upper limit, to 0.1 mm, preferably 0.25 mm, more preferably 0.5 mm, especially 0.7 mm, as lower limit.

The meshes of the net are preferably selected from the group of tetragonal, hexagonal, or octagonal meshes.

The tetragonal meshes are meshes in the form of a parallelogram with the sides a and b. The term “parallelogram” also, of course, encompasses the terms “rectangle” and “square”. The smaller angle between the two sides of the parallelogram is as a rule between 60 and 90°. In the borderline case of 90°, the parallelogram is a rectangle. In the borderline case where a=b and 90°, it is a square. The parallelogram additionally has a height hₐ. In the case of a rectangle or of a square, the height hₐ corresponds to the length of the side a. Square meshes are particularly preferred.

In the case of the hexagonal meshes, three pairs of sides a, b, and c, each parallel to one another, are arranged at distances hₐ, h_{b}, and h_{c}. In the case of the octagonal meshes, four pairs of sides a, b, c, and d, each parallel to one another, are arranged at distances hₐ, h_{b}, h_{c}, and h_{d}. The skilled person is aware that octagons cannot be used to produce continuous patterns. A net which includes octagonal meshes therefore also includes at least one second kind of meshes. These may be tetragonal meshes.

In one specific embodiment of the invention, the height hₐ for the parallelogram, the hexagon, and the tetragon is 0.1 to 0.99 mm, preferably 0.1 to 0.9 mm, more preferably 0.12 to 0.8 mm, and very preferably 0.25 to 0.7 mm.

For the parallelogram, the length-to-height ratio b/hₐ is 1:1 to 5:1, preferably 1:1 to 4:1, and more preferably 2:1 to 4:1. In the event of a ratio b/hₐ of 1:1, therefore, the meshes may comprise a square having a side length of 0.1 to 0.99 mm. For a larger ratio b/hₐ, the structure is elongated in one direction. By virtue of the distance hₐ of not more than 0.99 mm, even relatively small insects are effectively prevented from passing through the net, while the length may well be greater than 0.99 mm, and so the air permeability of the net is not unduly hindered.

In the case of a hexagon, the ratio ((h_{b}+h_{c})/2)/hₐ is 1:1 to 5:1, preferably 1:1 to 4:1, and more preferably 2:1 to 4:1. The situation here is analogous to that of the parallelogram. In the event of a ratio of 1:1, the structure is a regular hexagon with three equal sides, each having the same distance of not more than 0.99 mm from one another. In the case of a larger ratio ((h_{b}+h_{c})/2)/hₐ, the hexagon is elongated in one direction. The effect with regard to passage of insects and air is as for the parallelogram.

In the case of an octagon, the ratio ((h_{b}+h_{c}+h_{d})/3)/hₐ is 1:1 to 5:1, preferably 1:1 to 4:1, and more preferably 2:1 to 4:1. The situation here is analogous to that of the parallelogram. In the event of a ratio of 1:1, the structure is a regular octagon with four equal sides, each having the same distance of not more than 0.99 mm from one another. In the case of a larger ratio ((h_{b}+h_{c}+h_{d})/3)/hₐ, the octagon is elongated in one direction. The effect with regard to passage of insects and air is as for the parallelogram.

As well as tetragonal and hexagonal meshes, it is also possible in this embodiment, for example, to employ combinations of tetragonal and octagonal meshes or to vary the shape and size of the meshes in parts of the net. For example, the edges of the net may be knitted more densely, or else relatively thick textile fibers, including those produced from a different polymer, may be knitted in at certain distances for the purpose of stabilization.

The terms “height” and “length” refer to the open area of each mesh without taking into consideration the fibers, or the coated fibers. Analogously, the term “mesh size” for the purposes of this invention denotes the hole size of the meshes, i.e., the open area of each mesh without taking into consideration the fibers, or the coated fibers.

Textile net materials according to this embodiment of the invention are described in WO 2010/012671.

The thickness of the fibers used for producing the textile material of the invention, more particularly the nets of the invention, is selected by the skilled person in accordance with the desired properties of the net. As a general rule, the thicker the fibers, the greater the mechanical stability of the net; on the other hand, the proportion of open area in comparison with the proportion of the fiber-covered area will decrease continuously with decreasing mesh size. As a general rule, the fiber thickness should be such that the net has an open area of at least 20%, preferably at least 40%, and more particularly at least 50%. Nets of the type outlined are available commercially.

The nets used may preferably be single-layer nets. However, they may also be what are known as spacer fabrics, where two nets are joined to one another by means of individual yarns, to form a double layer.

The term “impregnating” denotes any kind of treatment of the textile structure with at least one insecticide that achieves a homogeneous distribution of the mixture on or in the sheetlike structure. Homogeneous here means that the concentration of a particular insecticide is substantially the same at each point of the sheet.

In one embodiment, impregnation is accomplished by coating the sheetlike structure or, preferably, monofilaments or multifilaments or fibers from which the sheetlike structure is produced, with at least one insecticide together with a binder (variant A).

The function of the binder is to fix the insecticide on the monofilaments or multifilaments or fibers of which the sheetlike structure is produced, or on the prefabricated sheetlike structure (“end of line coating”) (described below by reference to a net, as an example). The effect of this is that the active ingredient cannot be leeched out, or at least only very slowly.

The polymeric binder may in principle comprise any desired binder, subject to the proviso that the binders are capable of fixing the insecticide mixture in particular on textile materials. Preference is therefore given to binders known from the field of textile impregnation and textile coating. As will be appreciated, a mixture of two or more different binders may also be used.

Examples include (meth)acrylic homopolymers or copolymers, polyurethanes, polyisocyanurates or waxes, such as polyethylene waxes.

The binders in question may be, for example, binders obtainable by polymerizing ethylenically unsaturated monomers, preferably at least one monomer from the group selected from (meth)acrylates, more particularly C₁- to C₁₂ esters of (meth)acrylic acid, (meth)acrylates containing crosslinking groups, (meth)acrylic acid, maleic acid or esters of maleic acid, acrylonitrile, styrene, vinyl acetate, vinyl alcohol, ethylene, propylene, allyl alcohol or vinyl chloride.

In one preferred embodiment of the invention, this is a copolymer of ethylenically unsaturated monomers which comprises, as monomers, 50 to 95% by weight of at least one (meth)acrylate (A) of the general formula H₂C═CHR¹—COOR², where R¹ is H or methyl and R² is an aliphatic, linear or branched hydrocarbon radical having 1 to 12 carbon atoms, preferably 2 to 10 carbon atoms. R¹ is preferably H. Examples of suitable radicals R² comprise in particular methyl, ethyl, n-butyl or 2-ethylhexyl radicals, preferably ethyl, n-butyl or 2-ethylhexyl radicals. Moreover, the copolymer comprises 1 to 20% by weight of (meth)acrylic acid or (meth)acrylic acid derivatives (B) with additional functional groups. This may take the form in particular of a (meth)acrylic ester and/or (meth)acrylamides. The functional groups serve to bind the binders to the nets and can furthermore be used for crosslinking. For example, they may take the form of ω-hydroxyalkyl (meth)acrylic esters, (meth)acrylic esters having epoxy groups such as, for example, glycidyl esters, (meth)acrylamides or derivatives thereof such as, for example, (meth)acrylic acid methylolamide of the formula H₂C═CH(CH₃)—CO—HN—CH₂—OH. It is at the same time possible to employ further ethylenically unsaturated, preferably monoethylenically unsaturated, monomers (C) which differ from A and B, for example acrylonitrile or styrene. As a rule, the amount of further monomers is from 0 to 30% by weight. Especially preferred is a binder which comprises 70 to 90% by weight of an acrylic ester of the formula H₂C═CH₂—COOR², where R² comprises 4 to 8 C atoms, and which is preferably n-butyl and/or 2-ethylhexyl, and furthermore 10 to 20% by weight of acrylonitrile, 1 to 10% by weight of (meth)acrylic acid or a (meth)acrylic acid derivative which has functional groups, in particular (meth)acrylic acid methylolamide.

The abovementioned preferred binders can preferably be prepared by methods known to the skilled person, preferably by means of emulsion polymerization.

Preferred is an acrylate binder, in particular a copolymer, obtainable by emulsion polymerization of the components B1 to B4, and optionally B5.

As component B1, one or more, preferably 1, 2 or 3, especially preferably one, (meth)acrylate(s) of the formula (I)

H₂C═CR¹—COOR² (I)

is/are employed, where the symbols have the following meanings:

R¹ is H or CH₃, preferably H, and

R² is C₁-C₁₀-alkyl, preferably methyl, ethyl, n-propyl, i-propyl, n-butyl, i-butyl, sec-butyl, t-butyl, n-pentyl, sec-pentyl, neopentyl, 1,2-dimethylpropyl, i-amyl, n-hexyl, i-hexyl, n-heptyl, n-octyl, 2-ethylhexyl, n-nonyl or n-decyl, especially preferably methyl, ethyl, n-butyl or 2-ethylhexyl, very especially preferred are ethyl, n-butyl or 2-ethylhexyl.

Preferred as component B1 are methyl acrylate, ethyl acrylate, n-butyl acrylate, 2-ethylhexyl acrylate, and methyl methacrylate. Also preferred are butyl acrylate on its own or in admixture with methyl methacrylate or ethyl acrylate. Especially preferred is n-butyl acrylate.

Substances which are employed as component B2 are at least one monomer from the group consisting of N-methylolacrylamide, N-methylolmethacrylamide, N,N′-bis-methylolmaleic diamide, and N,N′-bismethylolfumaric diamide.

Preferred are N-methylolacrylamide and N-methylolmethacrylamide, in particular N-methylolmethacrylamide.

Substances which are employed as component B3 are one or more monomers, preferably one or two monomers selected from the group consisting of acrylic acid, methacrylic acid, vinylsulfonic acid, maleic acid, and fumaric acid. Preferred are acrylic acid and methacrylic acid; acrylic acid is especially preferred.

Substances which are employed as component B4 are one or more monomers, preferably one or two monomers, selected from groups B4A and/or B4B.

Monomers of group B4A are those of the formula (II) and/or (III)

H₂C═CR³X (II)

ZHC═CHZ (III)

where the symbols have the following meanings:

R³ is H or CH₃, preferably H;

X is Z, —CO—NH—CH₂—NH—CO—CR³═CH₂ or COO—CH₂—CO—CH₂—COOR⁴, preferably Z;

Z equals CONH₂, CONH—CH₂—OR⁵, COO—Y—OH, COO-glycidyl, CHO, CO—Y—OH, preferably CONH₂;

Y is C₁-C₈-alkylene, preferably C₂-C₆-alkylene, and

R⁴, R⁵ are identical or different and are a linear or branched C₁-C₁₀-alkyl group;

and (meth)acrylic-modified benzophenones, as described, for example, in EP-A 0 346 734.

Preferred as monomers from group B4A are acetoacetyl acrylate, acetoacetyl methacrylate, acrylamide, methacrylamide, maleic diamide, N-methoxy-methylacrylamide, N-n-butoxymethylacrylamide, 3-hydroxypropyl acrylate, 3-hydroxypropyl methacrylate, 4-hydroxybutyl acrylate, 4-hydroxybutyl methacrylate, 6-hydroxyhexyl acrylate, 6-hydroxyhexyl methacrylate, 2-hydroxy-3-chloropropyl acrylate, 3-hydroxy-3-chloropropyl methacrylate, glycidyl acrylate, and glycidyl methacrylate. Especially preferred are acrylamide, 3-hydyroxypropyl methacrylate, butanediol monoacrylate acetylacetate, glycidyl methacrylate, and 4-acryloxy-benzophenone.

Substances which are employed as monomers from group B4B are allyl acrylate, methallyl acrylate, allyl methacrylate, methallyl methacrylate, diallyl maleate, dimethylallyl maleate, allyl fumarate, methallyl fumarate, diallyl phthalate, dimethylallyl phthalate, diallyl terephthalate, dimethallyl terephthalate, p-divinylbenzene, butane-1,4-diol diallyl ether, and butane-1,4-diol dimethylallyl ether.

Preferred monomers of group B4 are those of group B4A, the use of one or two monomers from among this group being preferred.

Preferred monomers of group B5 are those of group B5A, and also vinylaromatic monomers of group B5B.

It is preferred to employ acrylonitrile or methacrylonitrile, preferably acrylonitrile, as component B5A.

Preferred as component B5B are styrene and α-methylstyrene, styrene being especially preferred.

In a preferred embodiment, acrylonitrile is employed as monomer of component B5 for the preparation of the acrylate binder.

The acrylate binder (B) is obtainable by emulsion polymerization of (data in % by weight are in each case based on the total amount of B):

b1) 20 to 93% by weight, preferably 50 to 90% by weight, especially preferably 60 to 90% by weight, in particular 75 to 85% by weight, of component B1;

b2) 1 to 5% by weight, preferably 1.5 to 3% by weight of component B2;

b3) 0.2 to 5% by weight, preferably 0.5 to 4% by weight, especially preferably 0.75 to 4% by weight, in particular 1 to 3% by weight of component B3;

b4) 0 to 7% by weight, preferably 0 to 5% by weight, especially preferably 0 to 4.5% by weight, in particular 0 or 0.2 to 4.5% by weight of component B4 and

b5) 0 to 40% by weight, preferably 5 to 40% by weight, especially preferably 5 to 30% by weight, in particular 0 or 5 to 26% by weight of component B5.

Suitable processes are known to the skilled person and described, for example, in WO 2005/064072 (page 20, line 20 to page 23, line 15).

The weight-average molecular weight of the non-crosslinked emulsion polymers obtained is generally between 40 000 and 250 000 (as determined by GPC (gel permeation chromatography)). The molecular weight is generally adjusted by using chain termination reagents, for example organosulfur compounds, in the usual amounts.

The especially preferred acrylate binder is generally obtained in the form of an aqueous dispersion and is usually employed in this form in the insecticidal formulation according to the invention.

The preferred acrylate binder can further comprise usual additives known to the skilled person, as for example film formers and/or plasticizers, such as adipates, phthalates, butyl diglycol, mixtures of diesters, obtainable by reacting dicarboxylic acids with straight-chain or branched alcohols. Suitable dicarboxylic acids and alcohols are known to the skilled person.

Others which are suitable, apart from the abovementioned binders, are silicone oils and silicone waxes, polysiloxanes, resins with fluorinated hydrocarbon radicals, melamine/formaldehyde condensates, methylolurea derivatives and curable polyesters, with silicone oils being preferred.

The preferred silicone oils and silicone waxes generally take the form of linear or cyclic polyorganosiloxanes, preferably polyalkyl- and/or polyphenylsiloxanes, alkyl being for example methyl, ethyl, propyl or octyl, preferably methyl. Particularly preferred are polydimethylsiloxanes, poly(methylphenylsiloxanes) and corresponding compounds in which a proportion of the methyl groups is replaced by higher alkyl groups. The molecular weight is preferably between 1000 and 150 000. Optionally, the silicone oils and in particular the silicone waxes may comprise consistency regulators, for example metal soaps such as lithium stearate, highly disperse silica, PTFE, boron nitride or urea, in order to obtain a pasty or fatty consistency.

For producing the sheetlike structures of the invention, in particular nets, the binders may be employed in the form of a formulation in a solvent, preferably as an aqueous formulation. However, the invention also embraces the use of solvent-free formulations.

In a preferred embodiment, aqueous formulations are employed which comprise 55 to 99% by weight of water, preferably 85 to 98% by weight of water and 1 to 45% by weight, preferably 2 to 15% by weight, of solids, the quantities given being in each case based on the total of all components in the formulation. The precise concentration also depends on the adsorptivity of the textile substrate.

The solids take the form of at least one binder, the insecticidal mixture, optionally at least one crosslinker, and optionally further components.

It is preferred to employ at least one water-dispersible crosslinker. In particular in the case of the preferred acrylate binder, this may preferably take the form of a crosslinker which has free isocyanate groups. These may preferably take the form of isocyanurates which have free isocyanate groups, preferably isocyanurates which are derived from aliphatic, cycloaliphatic or aromatic diisocyanates having 4 to 12 carbon atoms. Examples comprise 1,6-hexamethylene diisocyanate, 1,12-dodecane diisocyanate, 2,2′- and 2,4′-dicyclohexylmethane diisocyanate or 2,4-tolyl diisocyanate. Preferred are isocyanurates based on 1,6-hexamethylene diisocyanate. Especially preferred are isocyanurates which have additional hydrophilic groups such as, in particular, polyethylene oxide groups. The preparation of such isocyanurates is known to the skilled worker. They are preferably employed as a solution in polar aprotic solvents such as, for example, ethylene carbonate or propylene carbonate. Further details on the preferred crosslinkers having isocyanate groups are disclosed in WO 2008/052913 page 34, line 6 to page 35, line 3. It is especially preferred to employ an isocyanurate which is based on 1,6-hexamethylene diisocyanate (HMDI) and which has additional polyethylene oxide groups, the isocyanurate being dissolved in propylene carbonate (70% by weight of HMDI in propylene carbonate). The free isocyanate groups amount to approximately 11 to 12% by weight based on the solution. The crosslinker is preferably employed in an amount of from 1 to 10% by weight based on the amount of all solids of the formulation. The isocyanurate-based crosslinkers are suitable especially for crosslinking the abovementioned copolymers. The formulation may further comprise typical additives and adjuvants, UV stabilizers, and colorants. Examples of such additives are mentioned in WO 2008/052913 page 35, line 17 to page 37, line 5.

Besides serving purely esthetic purposes, colorants and pigments may have a warning effect for example on birds or mammals, or may camouflage the insecticidal nets against insects. Moreover, dark colors may bring about shading, which may be desired, and may reduce the harmful effect of UV light on active ingredients and textile fibers when used in the open.

Wetting agents and thickeners may be employed to enable uniform coating with the treatment liquor of sheetlike structures which can only be wetted with difficulty, and therefore inhomogeneously, such as, for example, polyolefin fibers. For this purpose, it would also be possible to employ water-miscible solvents, which, however, is not preferred due to the harmful effect on the environment. A person skilled in the art is familiar with the adjuvants which are conventionally used and with their concentrations.

The formulations may preferably comprise antioxidants, peroxide scavengers, UV absorbers, and light stabilizers. This is particularly recommended in the case of nets which are exposed to increased UV irradiation in the open or in greenhouses. The abovementioned additives protect not only the substrate fibers, but also the active ingredients, from decomposition due to radiation.

Suitable UV absorbers are described for example in WO 02/46503 or in WO 2007/077101. UV absorbers may firstly be used as a component in the formulation for impregnating; secondly, they may also be incorporated as early as during the production of the fibers, as for example in the case of polyolefins and polyesters. It is also possible advantageously to employ mixtures of a plurality of stabilizers which have different protective effects. As a rule, from 0.2 to 5% by weight, preferably from 0.25 to 4% and very especially preferably from 0.5 to 3.5% by weight of stabilizer is employed based on the weight of the untreated net. The amount in the formulation will be adjusted by the skilled person accordingly.

In a further embodiment, impregnating takes place by admixing at least one insecticide to a polymer and jointly extruding polymer and at least one insecticide to form a monofilament, which is processed to form the sheetlike structure of the invention (variant B).

In a further embodiment of the invention, impregnating is carried out by directly incorporating at least one insecticide into a monofilament which is processed for example to give fibers, of which the sheetlike structure according to the invention consists or which are present therein. Preferably, the sheetlike structure in this variant is a net.

Suitable polymer material for the monofilament into which the mixture according to the invention can be incorporated are thermoplastic polymers, preferably those based on olefinically unsaturated monomers, for example polyolefins, polyvinyl chloride, polyvinyl alcohols, poly(meth)acrylates, but also polyesters and polycarbonates, and, if desired, mixtures of the abovementioned polymers with each other or with thermoplastic elastomers. Especially preferred are polyethylene, for example low-density polyethylene (LDPE), such as linear low-density polyethylene (LLDPE), ultra-low density polyethylene (ULDPE), medium-density polyethylene (MDPE) and high-density polyethylene (HDPE), polyethylene resins such as copolymers of ethylene and alpha-olefins with at least three C atoms, polypropylene homopolymers, random copolymers and block copolymers of propylene and alpha-olefins with four and more C atoms, copolymers of ethylene with unsaturated carboxylic acid compounds, for example poly(ethylene/methyl methacrylate), poly(ethylene/vinyl acetate) or poly(ethylene/acrylic acid), and mixtures of such polymers and copolymers. Examples of thermoplastic elastomers comprise olefin- and styrene-based thermoplastic elastomers. Preferred are copolymers with ethylene or propylene as the main component, but also block copolymers comprising polystyrene and polyisoprene and/or polybutadiene blocks, and hydrogenated derivatives of such copolymers.

To produce the monofilaments, an insecticide and the polymer may be mixed by melt-kneading. It is also possible first to prepare a masterbatch by melt-kneading suitable amounts of insecticide and polymer, which masterbatch is subsequently diluted to the desired concentration by melt-kneading with a further quantity of polymer. If the masterbatch method is employed, it is also possible to use different polymers for the masterbatch and for the subsequent dilution, for example an LLDPE for the masterbatch and an HDPE for diluting the masterbatch.

Besides the polymer and the insecticide, the polymer composition comprises, optionally, a pulverulent carrier material, preferably from the group of the talcs, kaolin, loarns, finely pulverulent SiO₂, carbon and dextrins. The pulverulent carrier material, if present, amounts to preferably from 0.01 to 10% by weight. The pulverulent carrier material can be mixed with the insecticide mixture and the polymer by melt-kneading, but it is preferred first to mix the insecticide mixture and the pulverulent material and subsequently to mix this mixture with the polymer, for example by melt-kneading. It is especially preferred to use a mixture of the pulverulent material and the insecticide mixture for preparing a masterbatch.

Besides polymer, insecticide, and, if desired, pulverulent carrier, the polymer composition comprises, if desired, customary additives to thermoplastic molding compositions, such as pigments, antioxidants, lubricants, etc.

To produce the filaments according to these embodiments, a mixture is prepared of, for example, polymer, insecticide, and, if desired, further additives by melt-kneading, preferably at elevated temperatures, the mixture is extruded and the extrudate is processed to give pellets. Such pellets can be drawn by melt-spinning, by the extrusion method, to give a filament from which nets according to the invention can be woven, for example by the Raschel method.

Details on netting material and its production for this embodiment of the invention are described for example in WO 2008/004711.

Insecticides contemplated include all known insecticides. Suitable insecticides are more particularly the following, already known active ingredients (see “The Pesticide Manual”, 2003 edition, E. C. Tanlin):

1) Acetylcholinesterase (AChE) inhibitors

carbamates, examples being alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, and triazamate.

Organophosphates, examples being acephate, azamethiphos, azinphos(-methyl, -ethyl), bromophos-ethyl, bromfenvinfos (-methyl), butathiofos, cadusafos, carbophenothion, chlorethoxyfos, chlorfenvinphos, chlormephos, chlorpyrifos(-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemetonmethyl, parathion(-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon and vamidothion.

2) Sodium channel modulators/voltage-dependent sodium channel blockers pyrethroides, examples being acrinathrin, allethrin (d-cis-trans, d-trans)ₛ beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta), cyphenothrin, deltamethrin, empenthrin, (IR-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (IR-transisomer), prallethrin, profluthrin, protrifenbute, pyresmethrin, resmethrin, RU 15525, silafluofen, tau-fluvalinate, tefluthrin, terallethrin, tetramethrin(-IR-isomer), tralomethrin, transfluthrin, ZXI 8901, and pyrethrines (pyrethrum);

DDT

oxadiazines, an example being indoxacarb.

3) Acetylcholine receptor agonists/antagonists

chloronicotinyls, examples being acetamiprid, clothianidin, dinotefuran, imidacloprid, nitenpyram, nithiazine, thiacloprid, thiamethoxam, nicotine, bensultap, and cartap.

4) Acetylcholine receptor modulators

spinosynes, an example being spinosad

5) GABA-gated chloride channel antagonists

organochlorines, examples being camphechlor, chlordane, endosulfan, gamma-HCH, HCH, heptachlor, lindane, and methoxychlor,

fiproles, examples being acetoprole, ethiprole, fipronil, pyrafluprole, pyriprole, and vaniliprole.

6) Chloride channel activators

mectines, examples being avermectin, emamectin, emamectin-benzoate, ivermectin, and milbemcycin.

7) Juvenile hormone mimetics, examples being diofenolan, epofenonane, fenoxycarb, hydroprene, kinoprene, methoprene, pyriproxifen, and triprene.

It is preferred to use insecticides which are active against wood pests, more particularly against beetle species which breed in wood and bark. These include, for example, insecticides such as the pyrethroids (e.g., alpha-cypermethrin). Also suitable are fipronil, amidrazone, and chiorfenapyr. Particularly preferred insecticides are alpha-cypermethrin, deltamethrin, and permethrin, fipronil, amidrazone, and chlorfenapyr. Especially preferred insecticides are alpha-cypermethrin, deltamethrin, and permethrin. A specially preferred insecticide is alpha-cypermethrin. In another specially preferred embodiment, the insecticide is chlorphenapyr.

The sheetlike structure is impregnated with an insecticide, as for example with one, two or three insecticides. It is preferably impregnated with one or two insecticides, especially with one insecticide. Besides at least one insecticide, the sheetlike structure may comprise further pesticides, such as fungicides, herbicides, and/or growth regulators.

The amounts of insecticide per square meter of sheetlike structure are guided by the desired duration of activity and the strength of effect of the insecticide, and may be adjusted by the skilled person. The amount may be from 0.1 to 1000 mg/m². The amount of chlorfenapyr is usually 50 to 150 mg/m², preferably 70 to 130 mg/m², more preferably 90 to 110 mg/m². The amount of alpha-cypermethrin is usually 50 to 150 mg/m², preferably 70 to 130 mg/m², more preferably 90 to 110 mg/m². The amount of deltamethrin is usually 15 to 45 mg/m², preferably 20 to 40 mg/m², more preferably 25 to 35 mg/m². The amount of permethrin is usually 50 to 750 mg/m², preferably 75 to 650 mg/m², more preferably 100 to 550 mg/m². The amount of lambda-cyhalothrin is usually 5 to 30 mg/m², preferably 7.5 to 25 mg/m², more preferably 10 to 20 mg/m². If chlorphenapyr is used as a mixture with a pyrethroid, the chlorfenapyr: pyrethroid ratio is generally 0.06-30:1, preferably 0.1-10:1, more preferably 0.1-5:1.

The particle size of the insecticides in the aqueous formulation is generally from 50 nm to 20 μm, preferably 50 nm to 8 μm, more preferably 50 nm to 4 μm, more particularly 50 nm to 500 nm.

The sheet material may further comprise, in addition to the insecticide, at least one insect repellent. Suitable insect repellents are, for example, N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexanediol, indalon, methylneodecanamide (MNDA), {+/−)-3-allyl-2-methyl-4-oxocyclopent-2-(+)-enyl (+)-transchrysanthemate (esbiothrin), limonene, eugenol, (+)eucamal-1-ol, (−)-1-epi-eucamalol, extracts of Eucalyptus maculata, Vitex rotundifolia, Cymbopogan martinii, Cymbopogan citratus, Cymopogan nartdus, ethyl butylacetylaminopropionate or icaridin (1-piperidinecarboxylic acid 2-(2-hydroxyethyl)-1-methylpropyl ester).

The insect repellents and the further pesticides may be applied, for example, to the sheetlike structure beforehand, as for example by spraying of a textile material (with Fendona® from BASF SE, for example, a spray comprising alpha-cypermethrin). It is also possible, however, to apply these components only in situ (for example, with Fendona® Dip-it-yourself from BASF SE, a preparation comprising alpha-cypermethrin for impregnating nets made of textile fibers; or with K-O TAB® 1-2-3 from Bayer Crop Science, a preparation comprising deltamethrin for impregnating nets of textile fibers).

Sheetlike structures impregnated with an insecticide are available commercially, especially nets of textile fibers. Examples are Interceptor® from BASF SE (polyester net impregnated with alpha-cypermethrin), DuraNet® from Clarke Moquito Control Products Inc. (polyethylene net impregnated with alpha-cypermethrin), Dawa Plus® from Tanna Netting Co. Ltd (polyester net impregnated with deltamethrin), NetProtect® from Bestnet Europe Ltd (polyethylene net impregnated with deltamethrin), Iconet® from Syngenta AG (polyester net impregnated with lambda-cyhalothrin), Olyset® from Sumitomo Chemical (polyethylene net impregnated with permethrin), and Lifenet® from Bayer Crop Science AG (polypropylene net impregnated with deltamethrin).

The term “harmful insects” in accordance with the invention encompasses not only insects in the true sense but also damaging arachnids (Arachnida) responsible in particular as a vector for the transmission of diseases.

Examples of harmful insects are insects (Insecta) from the orders Diptera, Siphonaptera, Blattaria, Blattodea, Dermaptera, Hemiptera, Hymenoptera, Orthoptera, Isoptera, Thysanura, Phthiaraptera, Araneida, Lepidoptera, Coleoptera, and Acarina, and also from the classes Chilopoda and Diplopoda. Preferred are harmful insects from the orders Diptera, Hemiptera, Hymenoptera, Acarina, Lepidoptera, Coleoptera, and Siphonaptera. Especially preferred are harmful insects from the orders Lepidoptera and Coleoptera.

Examples of harmful insects are as follows:

Insects of the order Lepidoptera, such as Agrotis ypsilon, Agrotis segetum, Alabama argfflacea, Anticarsa gemmatalis, Argyresthia conjugella, Autographa gamma, Bupalus piniarius, Cacoecia murinana, Cameraria ohridella, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Estigmene acrea, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera satella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubfialis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Thaumatopoea processionea, Tortrix viridana, Trichoplusia ni, and Zeiraphera canadensis.

Beetles, (Coleoptera), such as Agrilus sinuatus, Agrilus viridis, Agrilus bigutaffus, Agrilus planipennis, Agriotes lineatus, Agriotes obscurus, Amphimallus solstitialis, Anisandrus dispar, Anoplophora glabripennis, Anoplophora chinensis, Anoplopophora sp., Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus, and Sitophilus granaria,

Flies, mosquitoes (Diptera), such as Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprin, Lucilia cuprina, Lucilla sericata, Lycoria pectoralis, Mansonia tillanus, Mayetio/a destructor, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus Tipula oleracea, and Tipula paludosa

Thrips (Thysanoptera), such as Dichromothrips corbetti, Dichromothrips ssp, Frankliniella fusca, Frankliniella occidentalis, Frankliniella tritici, Scirtothrips citri, Thrips oryzae, Thrips palmi, and Thrips tabaci,

Termites (Isoptera), such as Calotermes flavicollis, Leucotermes fiavipes, Heterotermes aureus, Reticulitermes flavipes, Reticulitermes virginicus, Reticulitermes lucifugus, Termes natalensis, and Coptotermes formosanus,

Cockroaches (Blattaria—Blattodea), such as. Blattella germanica, Blattella asahinae, Periplaneta americana, Periplaneta japonica, Periplaneta brunnea, Periplaneta fuligginosa, Periplaneta australasiae, and Blatta orientalis;

True beetles (Hemiptera including Homoptera), such as Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrats, Solubea insulars, Thyanta perditor, Acydhosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Hyalopterus Pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicda, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Climex lectularius, Cimex hemipterus, Reduvius senilis, Triatoma spp., and Arilus critatus.

Ants, bees, wasps, plant wasps (Hymenoptera), such as Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminate, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

Crickets and grasshoppers (Orthoptera), such as Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

Arachnoidea, such as arachnids (Acarina), examples being those of the families Argasidae, Ixodidae, and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricins, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora, and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g., Latrodectus mactans, and Loxosceles reclusa,

Flees (Siphonaptera), such as Ctenocephalides felis, Ctenocephalides canis, Xenopsylla cheopis, Pulex irritans, Tunga penetrans, and Nosopsyllus fasciatus,

Silverfish, firebrats (Thysanura), such as Lepisma saccharina and Thermobia domestica,

Centipedes (Chilopoda), such as Scutigera coleoptrata,

Millipedes (Diplopoda), such as Narceus spp.,

Earwigs (Dermaptera), such as Forficula auricularia,

Lice (Phthiraptera), such as Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus, and Solenopotes capillatus,

Nematodes such as Heterodera glycines, H. avenae, H. schachtg H. trifolii, H. gottingiana, H. cajani, H. zeae, Globodera rostochiensis, G. pallida, G. tabacum., Meloidogyne arenaria, M. incognita, M. javanica, M. hapla, M. chitwoodi, Ditylenchus destructor, D. dipsaci, D. angustus, Anguina tritici, A. agrostis, Afrina/Anguina wevelli, Pratylenchus penetrans, P. brachyurus, P. coffeae, P. zeae, P. goodeyi, P. thornei, P. vulnus, Radopholus Hirschmanniella oryzae, H. mucronata, H. spinicauda, Hoplolaimus columbus, H. seinhorsti, H. indicus, Rotylenchulus reniformis, Tylenchulus semipenetrans, Helicotylenchus multicinctus, H. multicinctus, H. mucronatus, H. dihystera, H. pseudorobustus, Criconemella C. xenoplax axestis, C. spharocephalum.

In a preferred embodiment, harmful insects of the order Lepidoptera are those of the family Gracillariidae, especially the subfamilies Thaumetopoeinae, Cossinae, Zeuzerinae, Larentinae, and Ennominae. Particularly preferred harmful insects of the order Lepidoptera are oak processionary (Thaumetopoea processionea), pine processionary (Thaumetopoea pityocampa), goat moth (Cossus cossus), leopard moth (Zeuzera pyrina), gypsy moth (Lymantria dispar), horse-chestnut leaf miner (Cameraria ohridella), oak leafroller (Tortrix viridana), winter moth (Operophtera brumata), and mottled umber (Erannis defoliaria).

In a preferred embodiment, harmful insects of the order Coleoptera are those of the families Curculionidae, Bupraestoidae, Cerambycidae, Scarabaeidae, and Hemiptera, especially the subfamilies Scolytinae, Molytinae, Rhynchophorinae, Agrilinae; Buprestinae, Lamiinae, Melonthinae, and Tinginae. Particularly preferred harmful insects of the order Coleoptera are European spruce bark beetle (Ips typographus), oak bark beetle (Scolytus intricatus), large elm bark beetle (Scolytus scolytus), six-spined spruce bark beetle (Pityogenes chalcographus), great spruce bark beetle (Dendroctonus micans), mountain pine beetle (Dendroctonus ponderosae), large brown pine beetle (Hylobius abietis), green weevil (Phyllobius spec.), red palm weevil (Rhynchophorus ferrugineus), beech splendor beetle (Agrilus viridis), jewel beetles (Agrilus spec.), oak jewel beetle (Coraebus undatus), flat-faced longhorns (Saperda spec.), Asian longhorn beetle (Anoplophora glabfipennis), citrus longhorn beetle (Anopolophora chinensis), great capricorn beetle (Cerambyx cerdo), pine sawyer (Monochamus galloprovincialis), sawyer beetles (Monochamus spec.), forest cockchafer (Melolontha hippocastani), common cockchafer (Melolontha melolontha), sycamore lace bug (Corythucha ciliata).

In one particularly preferred embodiment, the method of the invention is suitable for protecting living cork oaks against harmful insects, such as the cork oak bark beetle, by using a sheetlike structure impregnated with an insecticide to cover the surface of trunk and/or branch of the cork oaks.

The present invention further relates to living plants whose trunk, pseudostem, branch, root ball and/or root region surface is covered with a sheetlike structure impregnated with an insecticide.

The present invention additionally relates to a rectangular sheetlike structure (especially a net of textile fibers) impregnated with an insecticide and comprising a fastening means which, following tubular coverage of the surface of trunk, pseudostem or branch of a plant, permits a durable hold thereto.

The fastening means comprises, for example, an adhesive material, a ribbon, a cord or a hook-and-loop fastening, preferably a hook-and-loop fastening. The fastening means typically connects the adjacent corners when the sheetlike structure is in tube form.

The shape of the rectangular sheetlike structure is largely rectangular, such as square, for example, or trapezoidal. Usually, at least two opposite edges are largely parallel. The edges may each have a length of 1 cm to 10.0 m, preferably 5 cm to 5.0 m, and especially 10 cm to 2.5 m.

In one preferred embodiment (referred to as a “trunk-height net”), the sheetlike structure has a height of 30 cm to 5.0 m, preferably 50 cm to 3.0 m, and more preferably 80 cm to 2.2 m. The height is guided by the height to which the trunk is to be covered with the sheetlike structure. The length is typically made such that the sheetlike structure is able to cover the girth of the trunk, pseudostem or branch. It is usually 10 cm to 5.0 m, preferably 30 cm to 3.0 m, and more preferably 50 cm to 1.5 m.

In another preferred embodiment (“sleeve-shaped net”), the sheetlike structure has a height of 0.5 cm to 50 cm, preferably 2 cm to 30 cm, and more preferably 4 cm to 20 cm. The length is typically made such that the sheetlike structure is able to cover the girth of the trunk, pseudostem or branch. It is usually 10 cm to 5.0 m, preferably 30 cm to 3.0 m, and more preferably 50 cm to 1.5 cm.

The present invention further provides a sheetlike structure (especially a net of textile fibers), impregnated with an insecticide, in the form of a perforated sheet which has a continuous interruption between the outer and inner edges. The continuous interruption may take the form of a largely radial cut.

The shape of the perforated sheet can be circular, oval, square, rectangular, or similar to one of these shapes. The shape of the hole in the perforated sheet can be circular, oval, square, rectangular, or similar to one of these shapes. It is also possible for the hole to take the form of at least one cut (e.g., a cross-shaped cut).

In one preferred embodiment the perforated sheet and the hole are largely circular. The diameter of the hole in the perforated sheet is usually 0.1 cm to 2 m, preferably 10 cm to 1.0 m, and especially 30 cm to 80 cm. The diameter of the perforated sheet is usually 10 cm to 30 m, preferably 1.0 m to 20 m, and especially 2.0 m to 10.0 m. The diameter of the hole in the perforated sheet is of course smaller than the diameter of the perforated sheet.

In another preferred embodiment, the perforated sheet is largely rectangular. The edges may each have a length of 50 cm to 30 m, preferably 1.0 m to 20 m, and especially 2.0 m to 10.0 m.

In order to allow the perforated sheet to lie closely against a trunk or pseudostem, the edge of the perforation may be provided with an elastic material which optionally comprises a hook-and-loop fastening. The perforated sheet may be fastened in the soil with customary tent pegs or the like, in the region of the outer edge.

The present invention further provides for the use of the sheetlike structure of the invention for protecting living plants from harmful insects.

The present invention offers a host of advantages: Individual plants or particular parts of plants can be protected in a very targeted and locationally precise way against the harmful actions of the insects. There is no contamination of the environment, as in the case of spray application, for example. As a result, the environment is protected and application is now possible even where hitherto there have been restrictions on the application of insecticide, for the purpose of protecting water or protecting particular organisms. The plants are protected efficiently from penetration of the bark or the wood by living and/or breeding insects or those which feed on plant parts. The harmful insects are usually killed on contact with the net. As a result of the long-lasting effect, the method is less laborious and in the long term less expensive than regular spraying with insecticides. The sheetlike structure in the form of fibers of textile material is permeable to air, and so there is no adverse effect on the living plants.

### EXAMPLES

### Example 1

### Tree Trunk Covered with High Net

Working example 1 uses an Interceptor® net from BASF SE, impregnated with alpha-cypermethrin (Polyester net 75 denier, 155 mesh). The rectangular net has a length of 180 cm and a height of 160 cm. The net is wound around the trunk (approximately 30 cm in diameter) of a tree, covering the surface of the trunk from the ground to a height of 180 cm above the ground. The net is fixed on the trunk by a standard commercial adhesive tape, which is bonded around the trunk and its covering net at about 10 cm above the ground and 180 cm above the ground. Alternatively, instead of the adhesive tape, a cord, rubber band or ribbon (elastic or nonelastic) with hook-and-loop fastening may be used. Instead of the Interceptor® net, for example, it is also possible for a DuraNet®, Dawa Plus, NetProtect®, lconet®, Olyset®, or Lifenet® to be used.

### Example 2

### Tree Trunk Covered with Net in Sleeve Form

Working example 2 uses nets as in example 1. The rectangular net has a length of 20 cm and a height of 10 cm. The net is wound around the trunk (approximately 3 cm in diameter) of a tree, covering the surface of the trunk at a height of 20 cm (alternatively 60 or 160 cm) above the ground, in a width of 10 cm. The net is fixed on the trunk by a standard commercial adhesive tape. Alternatively, instead of the adhesive tape, it is possible to use a cord, rubber band or ribbon (elastic or nonelastic) with hook-and-loop fastening. As a further alternative, the net can be tied in sleeve form at the ends.

### Example 3

### Tree Trunk Covered with High Net Including Hook-and-Loop Fastening

In working example 3, the nets from example 1 are provided at the top and bottom edges with a strip, 2 cm wide and 100 cm long, of one side of a hook-and-loop fastening. At the corners at the top and bottom right, a strip, 2 cm wide and 10 cm long, of the opposing side of the hook-and-loop fastening is mounted in the region of the end of the 100 cm strip. This net can then be wound around the tree trunk. For fastening, all that need now be done is to contact the short end of the strip at the corners with the long strip, to close the hook-and-loop fastening. This ensures a secure hold on the tree trunk.

### Example 4

### Root Region Covered with Round Net

Working example 4 uses an Interceptor® net in the form of a perforated sheet having an external diameter of 5.0 m and a hole diameter of 10 cm, with a radial cut between the inner hole and the outer edge. The perforated sheet is placed around a tree trunk (approximately 15 cm in diameter) on the earth above the root region, with the trunk coming to lie in the region of the hole. In the region of the outer edge of the perforated sheet, approximately 10 tent pegs are inserted through the net into the soil for the purpose of fastening. Instead of the Interceptor® net it is also possible, for example, for a DuraNet®, Dawa Plus, NetProtect®, Iconet®, Olyset®, or Lifenet® to be used.

1-13. (canceled)

14. A method for protecting living plants from harmful insects comprising covering the surface of the trunk, pseudotrunk, branch, root ball and/or root region of the plant with a sheetlike structure impregnated with an insecticide.

15. The method according to claim 14, wherein the sheetlike structure is a textile material.

16. The method according to claim 14, wherein the harmful insects are of the orders Lepidoptera and Coleoptera.

17. The method according to claim 14, wherein the plants are trees.

18. The method according to claim 14, wherein the plants are deciduous trees.

19. The method according to claim 14, wherein the insecticide comprises a pyrethroid, fipronil, amidrazone and/or chlorfenapyr.

20. The method according to claim 14, wherein the sheetlike structure covers the surface for at least one month.

21. A living plant whose trunk, pseudotrunk, branch, root ball and/or root region surface is covered with a sheetlike structure impregnated with an insecticide.

22. The plant of claim 21, wherein the sheetlike structure is a textile material.

23. The plant of claim 22, wherein the plants are trees.

24. The plant of claim 22, wherein the plants are deciduous trees.

25. The plant of claim 22, wherein the insecticide comprises a pyrethroid, fipronil, amidrazone and/or chlorfenapyr.

26. The plant of claim 25, wherein the sheetlike structure covers the surface for at least one month.

27. A rectangular sheetlike structure impregnated with an insecticide and comprising a fastening means which, following tubular coverage of the surface of trunk, pseudotrunk or branch of a plant, allows a permanent hold thereto.

28. The sheetlike structure according to claim 27, the fastening means comprising a hook-and-loop fastening which connects the adjacent corners when the sheetlike structure is in tubular form.

29. The sheetlike structure of claim 28, wherein the sheetlike structure is a textile material.

30. A sheetlike structure impregnated with an insecticide, in the form of a perforated sheet which has a continuous interruption between the outer and inner edges.

31. The sheetlike structure according to claim 29, wherein the diameter of the perforation in the perforated sheet is 0.1 cm to 2 m, and the diameter of the perforated sheet is 10 cm to 30 m.

32. The sheetlike structure of claim 31, wherein the sheetlike structure is a textile material.
